(19) United States
(12) Patent Application Publication
(10) Pub. No.: US 2007/0128650 A1
(43) Pub. Date: Jun. 7, 2007

(54) Solid phase RFLP-based SNP detection

(75) Inventors: Allan C.P. Schell, Fort Collins, CO (US); Erica L. Daniell, Fort Collins, CO (US)

Correspondence Address: THE MCCALLUM LAW FIRM, P. C., 685 BRIGGS STREET, PO BOX 929, ERIE, CO, 80516, US

(73) Assignee: Gene Check, Inc., Fort Collins, CO (US)

(21) Appl. No.: 11/634,316

(22) Filed: Dec. 4, 2006

Related U.S. Application Data
(60) Provisional application No. 60/741,749, filed on Dec. 2, 2005.

(51) Int. Cl. C12Q 1/68; C12P 19/34; C07H 21/04

(52) U.S. Cl. 435/6; 435/91.2; 536/25.32

(57) ABSTRACT

The invention is directed to a method for detection of single nucleotide polymorphisms utilizing a solid phase RFLP-based method of detection. The invention further relates to a method of detecting a single nucleotide polymorphism (SNP) comprising providing a sample test DNA molecule containing an SNP site of interest; detectably labeling the DNA molecule at or near each of its ends with different labels and containing the SNP site of interest; providing at least two different immobilization oligonucleotides capable of immobilization to a solid support; digesting the test DNA molecule with a restriction endonuclease, whose cut site contains the SNP site in at least one allele of the SNP site; separating strands of the test DNA molecule; annealing the strands to the immobilization oligonucleotides; and detecting the labels of DNA strands annealed to the immobilization oligonucleotide.

### TECHNICAL FIELD OF THE INVENTION

The invention relates to methods for the detection of single nucleotide polymorphisms. The invention more specifically relates to a solid phase RFLP-based method of detection of single nucleotide polymorphisms.

### BACKGROUND OF THE INVENTION

Progress in human molecular and medical genetics today depends, to an increasing extent, on the efficient and accurate detection of mutations and sequence polymorphisms, most of which result from single base substitutions and small additions or deletions. Such mutations are responsible for a large number of genetic diseases and may be inherited, arise de novo in the germline (sporadic diseases) or be acquired somatically (e.g. cancers). In addition, single nucleotide polymorphisms (SNPs) are being identified at an increasingly rapid pace and will play an increasingly important role in gene discovery, pharmacogenetics and in the study of genetic variation. The scientific challenge in working with single nucleotide mutations and polymorphisms in higher organisms is to distinguish the mutant or polymorphic sequence from the normal allele, a problem made significantly harder by the fact that these organisms are diploid and SNPs are most often heterozygous.

Several methods have been, and are being, developed for the detection and genotyping of SNPs. One of the oldest and most robust is Restriction Fragment Length Polymorphism (RFLP) analysis. RFLP utilizes bacterial restriction enzymes, which are sequence specific double stranded endonucleases. If one allele of a SNP creates or destroys a restriction endonuclease cut site, the alleles can be distinguished by amplifying the region of DNA in which the SNP resides by Polymerase Chain Reaction (PCR), digesting the amplicon with the appropriate restriction endonuclease and running the digested product on an acrylamide or agarose gel. If a single large band is observed, the sample is homozygous for the genotype without the cut site. If two bands are observed, the sample is homozygous for the genotype containing the cut site. If three bands are observed, one at the position of uncut amplicon and the other two at the positions of the cut fragments, the sample is heterozygous for the cut site i.e., SNP.

The large number of available restriction endonucleases and the ease and robustness of RFLP analysis has made it a very popular method for genetic analysis. RFLP is also a preferred method of species and individual identification. However, RFLP analysis initially suffered from two major drawbacks in that all SNPs do not create or destroy restriction endonuclease cut sites and the requirement of gel electrophoresis for genotyping.

It has been shown that RFLP sites can be created by using primers ending adjacent to the SNP site and containing a mismatch close to the SNP site that, following amplification, converts the sequence of one allele of the SNP to a restriction endonuclease site (Haliassos et al, 1989, Nucleic Acids Research 17:3606). This greatly expands the utility of RFLP analysis but it still requires gel electrophoresis and, currently, conventional RFLP is not suitable for multiplexing, i.e., the simultaneous analysis of multiple SNPs.

If RFLP can be adapted to a high throughput platform, such as flow cytometry, its popularity and utility will certainly increase. Flow cytometry is a powerful technology for multi-parameter analysis of particles or cells in solution. Flow cytometry utilizes fluorescent molecules (fluors) and the scatter of laser light to obtain rapid analysis of particles in solution. Flow cytometers can detect particles flowing past a laser beam by means of the light scattering caused by the particles or by detecting fluorescent light emitted by a particle when it is labeled with fluorescent molecules. The intensity of the fluorescent light detected is proportional to the number of fluorescent molecules per particle in the light source and thus allows quantitative information to be gathered about the extent of particle labeling.

Fluorescent molecules are excited by light of one wavelength and emit light of another wavelength. A wide variety of fluors are available commercially and can be attached to synthetic DNA oligonucleotides. These fluorescently labeled DNA oligonucleotides can then be used as primers in PCR to create fluorescently labeled PCR fragments. The ability of flow cytometers to detect the intensities of multiple fluors independently allows for simple multiplexing (Iannone et al, 2000, Cytometry 39:131-140). It is also possible to distinguish different sized microspheres (beads) by virtue of differences in their light scattering ability. There are currently bead sets available commercially that contain as many as 100 easily distinguished beads. Even more complex bead sets are under development to allow multiplexing of as many as 1000 assays simultaneously. The availability of bead sets allows for easy multiplexing in bead based flow cytometric assays. DNA oligonucleotides can be attached to an avidin coupled to beads. Attaching oligonucleotide sequences to beads is well understood and allows for specific annealing of DNA or PCR products to multiplexed bead sets.

Flow cytometery has been used to detect SNPs (Lee et al, 2004, Theor. Appl. Genet. 110:167-174). Comparisons of single base extension (SBE), allele specific primer extension (ASPE), direct hybridization (DH), and oligonucelotide ligation (OL) assays have all been performed using plant DNA. DH and OL have been shown not to work well for all SNPs. SBE and ASPE have been shown by the same authors to be successful, but rather expensive on a per sample basis. To date, no commercially successful flow cytometry-based RFLP genotyping method has been described.

Microarrays are a widely used and extremely popular platform for the analysis of DNA and RNA. Like flow cytometry, microarray technology is based on detection of fluorescent molecules. DNA microarrays consist of oligonucleotides which are covalently linked to the surface of slides, which are analogous to microspheres in flow. One slide may have many spots, each comprised of a different oligonucleotide. Slides can be formed with more than ten thousand spots. Fluorescently labeled DNA can be annealed to the oligonucleotides attached to a slide. The specificity of annealing sample DNA to immobilized oligonucleotides is based on sequence homology, and allows for high level multiplexing within one slide. The intensity of the fluorescent light detected at each spot is directly proportional to the number of fluors annealed to that spot. A large number of fluors that can be detected by microarray readers are available commercially, and, as with flow cytometry, the fluors can be ordered attached to PCR primers. Identical fluors can frequently be used in both microarray and flow cytometry applications. Microarray technology allows for analysis of highly multiplexed fluorescently labeled DNA samples. Genotyping has been demonstrated using microarray technology (Ji et al, 2004, Mutation Research 548:97-105). The standard method to distinguish SNPs is to use allele specific fluorescence hybridization. This method of SNP detection requires stringent annealing conditions, and may not work for all SNPs (Lee et al, 2004, Theor. Appl. Genet. 110:167-174).

An inexpensive and efficient method of detecting SNPs is greatly needed that allows high order multiplexing and can be easily adapted to a variety of platforms, including flow cytometry and microarray platforms.

### SUMMARY OF THE INVENTION

In one embodiment, the invention is directed to a method of detecting a single nucleotide polymorphism (SNP) comprising providing a sample test DNA molecule containing the SNP site of interest; detectably labeling the DNA molecule at or near each of its ends with different labels; providing at least two different immobilization oligonucleotides capable of immobilization to a solid support; digesting the test DNA molecule with a restriction endonuclease, whose cut site contains the SNP site in one allele of the SNP site; separating strands of the test DNA molecule; annealing the strands to the immobilization oligonucleotides; and detecting the labels of DNA strands annealed to the immobilization oligonucleotides.

In another embodiment, the invention is directed to a method for detecting a single nucleotide polymorphisms comprising obtaining a sample of a test DNA molecule; amplifying and detectably labeling both strands of the test DNA by PCR; digesting the PCR amplicon with a restriction endonuclease; annealing the test DNA to at least two different immobilization oligonucleotides capable of immobilization to a solid support such that, if a given molecule of the PCR amplicon contains an allele of the SNP that allows it to be digested by the restriction endonuclease, digestion by the restriction endonuclease will remove a reporter label from the strand annealing to one of the immobilization oligonucleotides; detecting the ratio of the reporter label to a control label to determine a SNP genotype.

### BRIEF DESCRIPTION OF THE DRAWING FIGURES

FIG. 1 is a schematic representation of solid phase RFLP according to an embodiment of the invention.

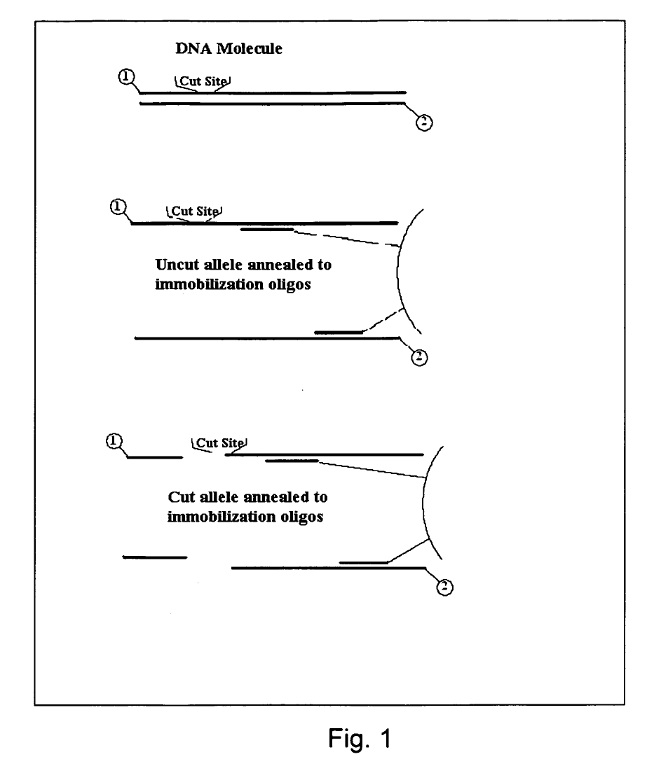

FIG. 2 is a graphic representation of the genotyping of sheep prion protein codon 136 by RFLP and flow cytometry according to an embodiment of the invention.

### DESCRIPTION OF THE INVENTION

Solid phase RFLP genotyping is a simple and robust method of SNP detection that greatly increases the power of RFLP analysis, allows high order multiplexing and can be easily adapted to a variety of platforms, including flow cytometry and microarray platforms.

In an embodiment of the invention, solid phase RFLP technology uses both strands of a sample DNA molecule to obtain more information about the sample. DNA strands are separately and distinguishably labeled, for example, with different fluorescent labels, wherein the labels are located on or near opposite ends of the target (test) DNA molecule. The DNA molecule is digested with a restriction endonuclease capable of cutting only one allele of the SNP. Following digestion, the DNA molecules are denatured and annealed to two immobilization oligonucleotides which are immobilized to a solid support, or are capable of being immobilized to a solid support. The immobilization oligonucleotides, or portions of the immobilization oligonucleotides, are complementary to opposite strands of the test DNA molecule, and are complementary to regions on the same side of the restriction endonuclease recognition sequence within the DNA molecule.

Following annealing and immobilization, labels are detected. The label on the strand labeled on the same side of the restriction enzyme cut site as the sequences complementary to the immobilization oligonucleotide is the control label, and will always be found annealed to one of the immobilization oligonucleotides, unless there has been some failure in a step of the assay. In addition, the control signal can be used as a measure of assay efficiency to normalize the results. The label on the end of the DNA molecule on the opposite side of the SNP site relative to the sequences complementary to the immobilization oligonucleotides will only be found on the strand annealed to the other immobilization oligonucleotide if the test DNA molecule did not contain the restriction enzyme cut site. The amount of test signal relative to control signal will be used to determine genotype of the test DNA sample. The ratio of the test signal to the control signal will be characteristic for homozygous and heterozygous genotypes, i.e., low and high ratios will be characteristic of homozygotes wherein both chromosomes contain or do not contain the RFLP sequence. Heterozygotes will produce an intermediate signal.

DNA molecules can be derived from any source including, but not limited to, genomic DNA of any species, viral DNA, plasmid DNA, PCR amplicons, restriction fragments, or cloned DNA. In one embodiment, DNA molecules will be PCR amplicons or restriction enzyme digestion fragments. It must be possible to label test DNA molecules with two different labels which are distinguishable and are at or near opposite ends of the DNA molecule. Such DNA molecules can be prepared using a pair of oligonucleotide primers, each modified at the 5′ end with a detectable label such that they can be quantitatively detected by appropriate detection methods. Alternatively, DNA molecules can be labeled enzymatically, for example by using terminal transferase to add labeled nucleotides to the 3′ ends. In one embodiment, the oligonucleotide is biotin-modified, and is detectable using a detection system based on avidin or streptavidin which bind with high affinity to biotin. The streptavidin can be conjugated to an enzyme, the presence of which is detected using a chromogenic substrate and measuring the color developed.

Examples of useful enzymes in the methods of the present invention include but are not limited to horseradish peroxidase, alkaline phosphatase, glucose-6-phosphate dehydrogenase, malate dehydrogenase, staphylococcal nuclease, delta-V-steroid isomerase, yeast alcohol dehydrogenase, .alpha.-glycerophosphate dehydrogenase, triose phosphate isomerase, asparaginase, glucose oxidase, .beta.-galacto-sidase, ribonuclease, urease, catalase, glucoamylase and acetylcholinesterase.

The detectable label can be a fluorophore or fluorescent label. When the fluorescently labeled molecule is exposed to light of the proper wavelength, its presence can then be detected due to fluorescence using microscopy or fluorometry. Fluorescent labels include, but are not limited to, Fluorescein (and derivatives), 6-Fam, Hex, Tetramethylrhodamine, cyanine-5, CY-3, allophycocyanin, Lucifer yellow CF, Texas Red, Rhodamine, Tamra, Rox, Dabcyl, phycoerythrin, phycocyanin, o-phthaldehyde and fluorescamine. DNA molecules may also be labeled with radioactive labels, digoxigenin, biotin, chemiluminescent labels or calorimetric labels. In one embodiment, the test DNA molecules are directly or indirectly labeled with fluorescent molecules. The test DNA may be of any length (up to an entire chromosome) and can be either genomic, viral or plasmid DNA, a PCR amplicon, or a restriction digestion fragment.

Immobilization oligonucleotides may be of any length. In one embodiment, they will be from about 20 to about 80 nucleotides long. In one embodiment, the oligonucleotides can be entirely complementary to a portion of the test DNA molecule. In another embodiment, the oligonucleotides can be only partially complementary to a portion of the test DNA molecule. Each immobilization oligonucleotide of a pair will have at least a portion of itself complementary to a portion of one strand of the test DNA molecule. The oligonucleotides of a pair, or portions of the oligonucleotides, will be complementary to regions, and in one embodiment, non-overlapping regions, on opposite strands of the test DNA molecule. These regions must be, at least partially, on the same side of the SNP site.

As used in the present invention, the oligonucleotide is immobilized to a solid support or carrier. By “solid support” or “carrier” is intended any support capable of binding the oligonucleotide. Well-known supports or carriers include, but are not limited to, natural cellulose, modified cellulose such as nitrocellulose, polystyrene, polypropylene, polyethylene, dextran, nylon, polyacrylamide, and agarose or Sepharose.RTM. Also useful are magnetic beads. The support material may have virtually any possible structural configuration. Thus, the support configuration can include microparticles, microarrays, beads, porous and impermeable strips and membranes, the interior surface of a reaction vessel such as test tubes and microtiter plates, and the like. In one embodiment, the support will include a bead. In another embodiment, the support will include a microarray slide. Those skilled in the art will know many other suitable carriers for binding the oligonucleotides or will be able to ascertain these by routine experimentation.

In one embodiment of the invention, samples are PCR amplified using primers labeled with two different detectable labels, e.g., fluorescent molecules, which labels can be attached to any of the bases of the primer. In one embodiment, attachment will be at the 5′ end. The PCR amplicons are digested by restriction endbnuclease specific for one allele of the SNP and samples are denatured and annealed to oligonucleotides immobilized to a substrate. In one embodiment the substrate will comprise one or more of a bead or a microarray slide. Each bead or array spot will have attached to them a large number of oligonucleotides of two different types. These two types of immobilization oligonucleotides will be complementary to opposite strands of the PCR amplicon, but will be complementary to regions on the same side of the restriction endonuclease recognition sequence within the PCR fragment. This will allow independent immobilization of both strands of the PCR amplicon to a support or carrier. Because the primers used to produce the amplicon are labeled with different fluorescent molecules, each strand of the PCR fragment will be labeled differently. One labeled strand will serve as a control for PCR amplification and as a standard by which to gauge the overall extend of amplification and amplicon labeling. The annealed portion of the control strand will always be labeled, regardless of whether it contains a RFLP site or not, and will, therefore, serve as an indicator of the amount of PCR product that has been immobilized. The other strand of the PCR fragment will serve as a reporter strand. The fluor from the reporter strand will only be immobilized if the PCR fragment is not cleaved by the restriction endonuclease. This assay eliminates the need for a baseline control for PCR amplification by introducing an internal control for PCR quantity. Homozygous samples should either be cut completely or not cut at all by the restriction endonuclease as indicated by a low ratio of reporter strand to control strand signal or high ratio of reporter to control strand signal respectively. Heterozygous samples will produce an intermediate ratio of reporter signal to control signal since only half of the heterozygous amplicon will be digested by the restriction endonuclease. This assay also eliminates the need to analyze samples by gel electrophoresis or mass spectroscopy, and is easily multiplexed.

If one of a pair or set of alleles of a given SNP does not create a restriction endonuclease cut site, such a cut may be introduced into the test DNA sequence during PCR amplification. It has been shown that RFLP sites can be created by using primers ending adjacent to the SNP site and containing a mismatch close to the SNP site that, following amplification, converts the sequence of one allele of the SNP to a restriction endonuclease cut site (Haliassos et al, 1989, Nucleic Acids Research 17:3606). One of skill in the art will be familiar with this technique.

Genotyping is accomplished by comparing the ratios of reporter signals to control signals. Because both immobilization oligonucleotides hybridize to the amplicon on the same side of the RFLP site, one (the control oligonucleotide) will always hybridize to a strand containing a fluorescent label and will provide a control for successful PCR amplification, i.e., no label bound to these oligonucleotides will indicate failure of amplification. The reporter oligonucleotide will bind label only if there are uncut molecules in the amplicon. The ratio of reporter signal to the control signal will be characteristic for homozygous and heterozygous genotypes, i.e., low and high ratios will be characteristic of homozygotes wherein both chromosomes contain or do not contain the RFLP sequence. Heterozygotes will produce an intermediate signal.

### EXAMPLE

In this experiment, a region of the sheep prion protein gene surrounding codon 136 was amplified. Two alleles of codon 136 have been reported (valine-GTC and alanine-GCC). DNA coding for the valine allele can be cut by the BspHI restriction endonuclease (recognition sequence TCATGA). The amplicon used in these experiments is 285 base pairs long and was produced by amplifying sheep genomic DNA with the following primers:

(SEQ ID NO 1)

5′ biotin - TGCAGCTGGAGCAGTGGTA

(SEQ ID NO 2)

5′ Fitc - CCACTCGCTCCATTATCGTG

Amplicons were obtained from sheep genomic DNA of all three possible codon 136 combinations, i.e., AA, AV, and VV. PCR was conducted in 50 μl reactions containing 10 mM Tris (pH 8.3), 3.5 mM MgCl₂, 25 mM KCl, 10 μg/ml BSA, 10% glycerol, 100 ng of each primer, 0.2 mM dNTPs (Bioline), 1.5 units AmpliTaq (Applied Biosystems) and 50-100 ng sheep genomic DNA. PCR conditions were 94° C. for 5 min to denature genomic DNA, followed by 32 cycles of 94° C. for 30 sec, 50° C. for 30 sec (annealing) and 72° C. for 60 sec (extension). The last cycle was followed by an additional extension period of 5 min at 72°.

Following PCR amplification, 10 μl aliquots of reaction mix were digested for 2.5 hrs at 37° C. with 2 units of BspHI (New England Biolabs, Ipswich, Mass.) in 20 μl reaction volume containing the restriction enzyme manufacturer's buffer.

10⁶ carboxy coated beads (BioRad, Hercules, Calif.) were suspended in 5 μl of 200 mM MES (pH 6.1). 20 pmol of each of the immobilization oligonucleotides (see box below) were added and the final volume adjusted to 8 μl with water. 1 μl of 30 mg/ml EDC (Pierce, Woburn, Mass.) was added. The mixtures were vortexed gently and incubated in the dark for 30 min at room temperature. An additional 1 μl of EDC was added to the mixture, which was again gently vortexed and incubated in the dark for 30 min at room temperature. Following incubation, 1 ml of 0.02% Tween 20 was added and the bead solutions were centrifuged at 5000 g for 3 min. The supernatant was removed and the beads were resuspended in 1 ml of 0.1% SDS and again centrifuged at 5000 g for 3 min. The supernatant was removed and the beads resuspended in 150 μl 1 mM Tris (pH 7.4), 0.1 mM EDTA. Final bead concentration was determined by counting in a Partec CyFlow ML flow cytometer.

Immobilization Oligonucleotides

Reporter Oligonucleotide:
5′-NH₂-TCTGTAGTACACTTGGTTGGGGTAACGGTACATGTTTTCACGA
TAGTAAC
(SEQ ID NO 3)
Control Oligonucleotide:
5′-CAGAACAACTTTGTGCATGACTGTGTCAACATCACAGTCAAGCAACA
CACA-NH₂
(SEQ ID NO 4)

The reporter oligonucleotide is complementary to the biotin labeled strand of the PCR amplicon. The control oligonucleotide is complementary to the Fitc labeled strand. Both oligonucleotides are complementary to the region of the amplicon between the BspHI site and the Fitc label.

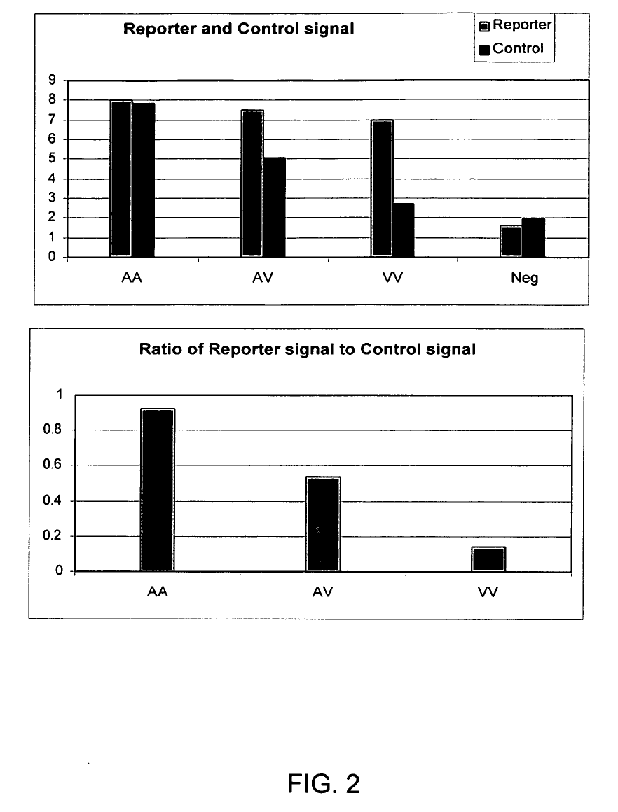

10 μl of BspHI digested PCR amplicon was added to 2000 beads (2 μl). The mixture was heated to 94° C. for 5 min (denaturation of PCR amplicon), 55° C. for 30 min (annealing of PCR amplicon to immobilization oligonucleotides) and cooled to 4° C. The beads were washed three times by adding 100 μl of TBST (50 mM Tris pH7.5, 150 mM NaCl, 0.05% Tween 20), gently mixing and pelleting the beads by centrifuging at 5000 g for 3 min. Following the third wash, the beads were resuspended in 50 μl TBST, 1% BSA ±100 ng Streptavidin/PE-Cy5 conjugate (Becton Dickinson, Bedford, Mass.) and incubated in the dark for 30 min at room temperature to allow binding of the Streptavidin/PE-Cy5 to the biotin label on the DNA. The entire bead mixture was added to 500 μl TBST and analyzed in the Partec CyFlow ML flow cytometer at a flow rate of 1 μl/sec. Beads were detected by forward scatter from the 488 nm laser and fluorescent channels were gated from the bead cluster in Forward Scatter vs. Side Scatter graph. Mean values for FL1 and Fl3 were obtained from Flow Max software. The data are presented in FIG. 2. Negative values were subtracted from signal values before calculation of ratios.

As can be seen by the data presented in FIG. 2, all possible genotypes of the codon 136 SNP alleles (AA, AV, VV) can easily be detected and distinguished, both from the raw data and from ratios of reporter label signal to control label signal.

While the invention has been explained in relation to specific embodiments, it is to be understood that various modifications thereof will become apparent to those skilled in the art upon reading the specification. Therefore, it is to be understood that the invention disclosed herein is intended to cover such modifications as fall within the scope of the appended claims.

What we claim is:

1. A method of detecting a single nucleotide polymorphism (SNP) comprising:
(a) providing a sample test DNA molecule containing an SNP site of interest;
(b) detectably labeling the DNA molecule at or near each of its ends with different labels and containing the SNP site of interest;
(c) providing at least two different immobilization oligonucleotides capable of immobilization to a solid support;
(d) digesting the test DNA molecule with a restriction endonuclease, whose cut site contains the SNP site in at least one allele of the SNP site;
(e) separating strands of the test DNA molecule;
(f) annealing the strands to the immobilization oligonucleotides; and
(g) detecting the labels of DNA strands annealed to the immobilization oligonucleotides.

2. The method of claim 1, wherein at least one allele, but not all alleles, of the SNP site in the test DNA molecule is included in a restriction endonuclease cut site.

3. The method according to claim 1, wherein the test DNA in the sample is amplified.

4. The method of claim 3, wherein the test DNA is a PCR amplicon or restriction digestion fragment.

5. The method of claim 1, wherein the immobilization oligonucleotides comprise from about 20 to about 80 nucleotides.

6. The method of claim 5, wherein the oligonucleotides of a pair, or portions of the oligonucleotides, will be complementary to regions on opposite strands of the test DNA molecule, which regions are at least partially, on the same side of the SNP site.

7. The method of claim 1, wherein the solid support comprises one or more of a microsphere, a bead, a microrray or a microtiter plate.

8. The method of claim 3, wherein the test DNA is PCR amplified using primers labeled with two different detectable labels, which labels can be attached to any base of the primer.

9. The method of claim 8, wherein the detectable labels comprise one or more of a fluorophore, a fluorescent molecule, a radioactive label, a chemiluminescent label, a colorimetric label, biotin or digoxigenin.

10. The method of claim 9, wherein the detectable label comprises a fluorescent molecule.

11. The method of claim 8, wherein the label on the strand wherein the label and the SNP site are on opposite sides of the sequence complementary to the immobilization oligonucleotide comprises a control label and the label on the strand wherein the label and the SNP site are on the same side of the sequence complementary to the immobilization oligonucleotide is a reporter label.

12. The method of claim 11, wherein the ratio of the reporter label in a strand annealed to one immobilization oligonucleotide to the control label in a strand annealed a second immobilization oligonucleotide is diagnostic of a SNP genotype.

13. The method of claim 1, wherein the step of detecting the labels of DNA strands annealed to the immobilization oligonucleotides comprises one or more of flow cytometry or microarray analysis.

14. A method for detecting a single nucleotide polymorphisms comprising:
(a) obtaining a sample of a test DNA molecule;
(b) amplifying and detectably labeling both strands of the test DNA by PCR;
(c) digesting the PCR amplicon with a restriction endonuclease;
(d) annealing the test DNA to at least two different immobilization oligonucleotides capable of immobilization to a solid support such that, if a given molecule of the PCR amplicon contains an allele of the SNP that allows it to be digested by the restriction endonuclease, digestion by the restriction endonuclease will remove a reporter label from the strand annealing to one of the immobilization oligonucleotides;
(e) detecting the ratio of a reporter label to a control label to determine an SNP genotype.

15. The method of claim 14, wherein the DNA strands are labeled using two oligonuceotide primers containing distinguishable detectable labels.

16. The method of claim 14, wherein the restriction endonuclease is specific for one allele of the SNP

17. The method of claim 14, wherein the immobilization nucleotides comprise from about 20 to about 80 nucleotides, and at least a portion of which is complementary to a portion of the test DNA molecule.

18. The method of claim 16, wherein the solid support comprises one or more of a microsphere, a bead, a microrray or a microtiter plate.

19. The method of claim 14, wherein the detectable labels comprise one or more of a fluorophore, a fluorescent molecule, a radioactive label, a chemiluminescent label, a calorimetric label, biotin or digoxigenin.

20. The method of claim 14, wherein the step of detecting the labels of DNA strands annealed to the immobilization oligonucleotides comprises one or more of flow cytometry or microarray analysis.


SEQUENCE LISTING (4 sequences)

SEQ ID NO: 1 - DNA, 19 bp, Artificial Sequence
  Artificially Generated Sequences
tgcagctggagcagtggta

SEQ ID NO: 2 - DNA, 20 bp, Artificial Sequence
  Artificially Generated Sequence
ccactcgctccattatcgtg

SEQ ID NO: 3 - DNA, 50 bp, Artificial Sequence
  Artificially Generated Sequence
tctgtagtacacttggttggggtaacggtacatgttttcacgatagtaac

SEQ ID NO: 4 - DNA, 51 bp, Artificial Sequence
  Artificially Generated Sequence
cagaacaactttgtgcatgactgtgtcaacatcacagtcaagcaacacaca